(19) United States
(12) Patent Application Publication
(10) Pub. No.: US 2010/0130993 A1
(43) Pub. Date: May 27, 2010

(54) Anastomosis device and system

(76) Inventors: Adrian Paz, Petach Tikva (IL); Gilad Heftman, Kibbutz Ein Gev (IL); Eitan Hod, Zichron Ya'akov (IL)

Correspondence Address: THE NATH LAW GROUP, 112 South West Street, Alexandria, VA, 22314, US

(21) Appl. No.: 11/989,242

(22) PCT Filed: Jul. 25, 2006
(86) PCT No.: PCT/IL2006/000862

§ 371 Date: Apr. 2, 2008

Related U.S. Application Data
(60) Provisional application No. 60/701,813, filed on Jul. 25, 2005.

(51) Int. Cl. A61B 17/11; A61M 29/00

(52) U.S. Cl. 606/153; 606/192

(57) ABSTRACT

An anastomosis device for forming two lumen-comprising body structures having an un-deployed and a deployed state and switchable from the former to the latter, comprising: a body with an overall cylindrical shape defining a proximal-distal extending axis of and comprising an external essentially cylindrical body part with a lumen and an internal body part at least a portion thereof being deployed within the lumen of the external body part and axially displacable with respect thereto in the switch between the un-deployed and the deployed states; said internal body part having an internal duct extending axially therethrough whereby in the deployed state there is a fluid communication between the distal and the proximal ends of the body; and two tissue-engaging units axially displaced from one another along said axis; at least one of the two units can change its state from a closed state in the un-deployed state of the device to an opened, tissue-engaging state in the deployed state of the device in which said first unit extends in an overall radial direction and is adapted in this state for tight association with internal walls of one of the body structures, such that the combined tissue-engaging action of the two unit holds the two structures together.

### FIELD OF THE INVENTION

This invention relates to a device and system for connecting two structures within the animal, particularly human body.

### BACKGROUND OF THE INVENTION

Anastomosis is a surgical connection between two structures in a mammalian body. It usually means making a connection between two tubular structures such as blood vessels or loops of the intestine, such as after a surgical procedure wherein a segment of the tubular structure(s) has been removed. Many devices have been devised for the anastomosis procedure. In some cases the two tubular structures have similar dimensions and in other cases, they have dissimilar dimensions.

Some relevant patents and published patent applications in the field include:

U.S. Pat. No. 4,911,164, to Roth, which describes a suture guide with a curved distal portion. The distal portion of the suture guide has a plurality of exterior axial grooves which can be used to align and guide a curved needle and attached suture. In order to drive the urethral stump to an accessible position, the device is provided with a plurality of outwardly extendable members which engage the lumen of the urethra. These members make it possible to push the urethral stump into approximation with the bladder neck.

U.S. Pat. No. 5,047,039, to Avant et al., describes a surgical device for the ligation of a dorsal vein and subsequent anastomosis. This device contains a pair of enclosed needles each having an attached suture which needles may be driven from the shaft of the device into adjacent tissue.

U.S. Pat. No. 5,591,179, to Edelstein, describes a suturing device including a shaft with portions defining an interior channel extending between a proximal and a distal end of the shaft. This channel includes a generally axial lumen which extends to the proximal end of the shaft and a generally transverse lumen which extends from the axial lumen distally outwardly to an exit hole at the outer surface of the shaft. A needle and suture can be back loaded into the transverse lumen of the channel while a generally non-compressible member can be movably mounted in the axial lumen of the channel. At the proximal end of the shaft a handle is provided with means operative to push the member distally through the lumen to deploy or expel the needle.

PCT Patent Application publication No. WO2004098417, to Scott, describes an anastomotic device for suturing together the urethral stump to the bladder neck. The device consists in an expandable flanged anchor that is introduced into the urinary bladder and one or more suture anchors that are passed into the urethral wall. Pulling on these sutures, which are connected to the bladder anchor, will approximate the urethral stump to the bladder. The sutures are fixed in position by an additional blocking member that is locked to the bladder anchor.

US Patent Application No. 2005/0070926 A1, to Ortiz, describes an applier for a fastener for a single lumen access anastomosis.

US Patent Application No. 2005/0165426 A1, to Manzo, describes a method and apparatus for anastomosis including annular joining member.

US Patent Application No. 2005/0165378 A1 to Heinrich et al., describes a method and apparatus for anastomosis including an expandable anchor.

US Patent Application No. 2005/0171564 A1 to Manzo, describes a method and apparatus for radical prostatectomy anastomosis.

US Patent Application No. 2005/0192602 A1 to Manzo, describes a method and apparatus for anastomosis including an anchoring sleeve.

US Patent Application No. 2005/0251155 A1, to Orban III, describes a method and apparatus for anastomosis.

US Patent Application No. 2005/0251175, to Weisenburgh II et al., describes anchors for use in attachment of bladder tissues to the pelvic floor tissues following a prostatectomy.

### SUMMARY OF THE INVENTION

The present invention provides a novel tissue-engaging device for connecting two internal lumens within the human body. Such a device will be referred to herein as an anastomosis device.

In accordance with the invention a novel anastomosis device is provided which includes a mechanical arrangement that can change from an un-deployed to a deployed state in which in the open state it engages tissue walls. The device of the invention has two tissue-engaging units.

The term “deployed state” refers to the state of the device in which is left in position in situ within the body, while the term “un-deployed state” refers to the original state of the device in which it is brought into position before being deployed. Thus, the device once in position within the body is switched from an un-deployed to a deployed state.

The invention provides, by one of its aspects, an anastomosis device having an un-deployed and a deployed state and switchable from the former to the latter, the device comprising: a body with an overall cylindrical shape defining a proximal-distal extending axis of and comprising an external essentially cylindrical body part with a lumen and an internal body part at least a portion thereof being deployed within the lumen of the external body part and axially displaceable with respect thereto in the switch between the un-deployed and the deployed states; said internal body part having an internal duct extending axially therethrough whereby in the deployed state there is a fluid communication between the distal and the proximal ends of the body; and two tissue-engaging units axially displaced from one another along said axis; at least one of the two units can change its state from a closed state in the un-deployed state of the device to an opened, tissue-engaging state in the deployed state of the device in which said first unit extends in an overall radial direction and is adapted in this state for tight association with internal walls of one of the body structures, such that the combined tissue-engaging action of the two unit holds the two structures together.

The term “having an overall cylindrical shape” denotes that the body has overall contours and side elevations resembling that of a cylinder. While the overall cylindrical shape may also include a perfect cylinder, it should be understood that in the fine details there may be deviations from the cylindrical shape. For example, the two tissue-engaging units, in their closed state, while fitting in the general cylindrical contour, may deviate therefrom in their fine details.

The switch from an un-deployed to a deployed state of the device is typically achieved, according to an embodiment of the invention, by axially displacing of the inner body part in a proximal direction with respect to the external body part (retraction).

According to an embodiment of the invention, the device may be removed after deployment for which purpose the device is switchable also from a deployed state to an un-deployed state. As will also be mentioned below, the actuation from a deployed into an un-deployed state may be achieved, according to an embodiment of the invention, through the employment of a release thread.

The external body part and the internal body part may be engaged to lock in the deployed state by a variety of mechanisms. As will be appreciated, the invention is not limited by the manner in which these two parts are locked with one another in the deployed state. For example, in accordance with an embodiment of the invention, the external body part has one or more first engaging members and the internal body part has one or more second engaging members. In the deployed state of the device the one or more first engaging members engage the one or more second engaging members. A specific example is a flap in one or both of these body parts which fits into and is biased to engage with opposite recesses in the other body part. A typical example are corresponding flaps and recesses in the distal side of these two body parts. Such flaps may typically be linked to a thread, which in the specific example of an anastomosis device used for an anastomosis of the bladder and the urethra, extends outside through the urethra or a catheter tube, such that when the thread is pulled the flap disengages the recess whereupon the two body parts are unlocked and the device can be switched from the deployed to an un-deployed state in which it can be removed. Another example is a ratchet-type engagement mechanism.

As will be appreciated there may be a variety of mechanisms that can be used for engaging and disengaging the two body parts one from the other and the invention is not limited to a specific manner in which this is achieved.

In accordance with another embodiment, the engaging members in the two body parts may be made from a biodegradable or a bioerodable material such that their respective biodegradation or bioerosion of these components releases the engagement. It should be pointed out that the device may at times also be entirely made from a biodegradable or a bioerrodable material.

In a specific, but not exclusive, embodiment of the invention at least one of the two tissue engaging unit, and preferably both, is an integral unit which is integrally formed within the external body part. An integral tissue-engaging unit is typically formed such that in its closed state it blends into the overall cylindrical shape of the external body part and it comprises engaging members that are each pivotally anchored to rigid portions of the external body part through a flexible connecting portion, and in an open state of the device extend in a general radial orientation.

The term “general axial orientation” is intended to denote that the engaging members are generally arranged in a proximal-distal fashion or define a general longitudinal axis which extends in said direction. Such an axis may at times be parallel to the device's axis although having a general proximal-distal orientation, may at times define the axis which is not fully parallel.

The term “general radial orientation” is intended to denote that in the open state the members are generally oriented in a direction away from the device's axis. In other words, a tangential cross-section through the device will display the members as extending radially.

In accordance with an embodiment of the invention, the integral unit comprises a plurality of ribs which in the closed state are folded and oriented in a general axial orientation and comprises a membrane that is fitted over either the internal faces (mainly the faces that point inward in the closed state of the tissue-engaging unit), or over the external faces.

The term “membrane” in the context of the invention, means to denote a sheet or a film, made of the variety of different materials which can form an essentially fluid-tight association with walls of organ or tissue. The membrane may be made of a variety water proof or water-tight materials such as plastic materials or other polymeric materials, rubber, woven or non-woven, typical impregnated fabric, etc. Exemplary materials are polyurethane, polypropylene, silicone and latex. In some embodiments of the invention, the membrane may incorporate or may be impregnated with an anti-microbial substance.

The membrane is typically made to at least partially cover the unit while in its open state, whereby in said open state such unit can form an essentially fluid-tight contact with the internal walls of the body structure in which it is deployed.

The membrane is typically made from a non-stretchable or minimally stretchable material and is attached to each one of the members (whether in the form of ribs or other) and is sized such that in the open state of the tissue-engaging unit it is stretched to its maximum thus conferring axial and tangential stability and rigidity to the tissue-engaging unit.

The term “fluid-tight contact” means to denote that there will be essentially no fluid flow between the membrane and the juxtaposed tissue. It should however be noted that the term “fluid-tight contact” does not mean that there is a hermetic seal that avoids flow of any fluid; rather that such flow would be relatively small and usually negligible.

An anastomosis device with an engaging unit that incorporates a membrane is novel per se and constitutes an aspect of the invention. In accordance with this aspect there is provided an anastomosis device having an un-deployed and a deployed state and switchable from the former to the latter, the device comprising: a body with an overall cylindrical shape defining a proximal-distal extending axis and two tissue-engaging units axially displaced from one another along said axis; at least one of the two units can change its state from a closed state in the un-deployed state of the device to an opened, tissue-engaging state in the deployed state of the device in which said first unit extends in an overall radial direction and is adapted in this state for tight association with internal walls of one of the body structures, such that the combined tissue-engaging action of the two unit holds the two structures together; said at least one unit having a membrane associated therewith such that in the tissue engaging state said membrane forms a fluid-tight contact with internal walls of the body structure. The body has preferably a structure with an internal and an external body parts, of a kind defined above. The at least one of the tissue-engaging units is preferably integrally formed with said body and typically with the external part thereof, of a kind specified above.

In one specific embodiment of the invention the external body part has one or two sections that define an integrally formed tissue-engaging unit. Each such unit comprises a plurality of engaging members each of which has a proximal and a distal segment which is linked to the external body part through respective proximal and distal flexible connecting portions, and linked to one another through an intermediate flexible connecting portion. The internal body part, according to this embodiment, has a portion that engages the distal end of the external body part in a manner such that axial displacement of the internal body part towards the proximal end with respect to the external body part exerts an axial force on the distal end and thus gives rise to an axial displacement in the same direction. Upon such displacement, the members then radially open into an open state in which the two segments are angled with respect to one another about the intermediate connecting portion and are each oppositely angled with respect to the external body part about the respective proximal and distal connecting portions. In a typical, although not exclusive example, the tissue-engaging units blend, in their closed state, into the overall cylindrical shape of the external body part.

In a specific embodiment of the integral tissue-engaging unit, the unit has a structure that comprises a plurality of elongated members that extend in a general axial orientation. Each of such members comprises a proximal and distal segment linked to one another through an intermediate flexible connecting portion at their respective distal and proximal ends. Thus, in an open state the two segments define an overall triangular shape with an apex that is constituted by the intermediate connecting portion. Such a structure is typically fitted with a membrane over an internal or external face, in a manner as described above. In some embodiments of the invention, the two engaging units have each such a structure, with the two units being either identical or being of a different size or shape.

In accordance with one embodiment of the invention, the tissue-engaging units have an essential axial symmetry.

In accordance with an embodiment of the invention, at least one engaging unit has a plurality of members with a portion that ends with a pointed, tissue penetrating tip, which in the open state of the device defines a prong that associates with internal walls of a body structure by penetrating in the layers thereof. Such a tissue-engaging unit is typically designed so that the prongs will be slanted, with either a general proximal or distal orientation. A specific example is an engaging unit, integrally formed within the external body part, and which has a plurality of members each of which has one segment that is linked at a point thereof between its proximal and distal ends to the other segment, said one segment having a free end with a pointed, tissue penetrating tip. Thus in the open state, the free ends assume an outwardly slanted orientation and can associate with the internal walls of the body structure as aforesaid. When said one segment is attached to the proximal segment, in accordance with a preferred embodiment, in the open state the free end points in a general distal direction (with a slant).

An anastomosis device which is characterized in that one of the tissue-engaging units has prong-like portions that can penetrate a tissue, is novel per se and an aspect of the invention. In accordance with this aspect, there is provided an anastomosis device with an un-deployed and a deployed state that is switchable from the former to the latter, and comprises: an essentially cylindrical body defining a proximal-distal extending axis of and having an internal duct extending axially therethrough providing fluid communication between the distal and the proximal ends of the body; an actuation mechanism for switching between the un-deployed and the deployed state of the device; and two tissue-engaging units axially displaced from one another along said axis; at least one of the engaging units has a plurality of members each comprising a segment having a free end with a pointed, tissue-penetrating tip, in the un-deployed state of the device said members blend into the cylindrical shape of the body, and in the deployed state, said free ends have an outwardly slanted orientation and can associate with internal walls of a body structure by penetrating inner layers thereof. In the device according to this aspect, the tissue engaging unit is preferably one that is integrally formed in the external body part, of the kind descried above. Further by some embodiments, the at least one tissue-engaging unit of this embodiment comprises a membrane of the kind described above.

A preferred, although not exclusive, use of the device of the invention is in linking two lumens, e.g. two cut stumps of organ that need to be connected to one another, e.g. following a surgery or an injury. A typical example is to connect the bladder with the urethral stump, following a prostatectomy procedure. In case of linking two lumens, there is usually a need to ensure flow communication between the two linked body portions and internal duct of the device provides such flow communication. Other possible uses of the device in accordance with the invention is in the anastomosis of two cut segments of the gastrointestinal tract or two cut segments of a blood vessel.

The invention also provides an anastomosis system that comprises a deployment instrument and device as described above. According to a preferred embodiment of the invention, the deployment instrument comprises a tubular body that after deployment of the device remains attached thereto and serves for catheterization. A typical deployment instrument comprises: an external tubular sleeve having a lumen which in the un-deployed state accommodates the device in its distal end; an inner sleeve accommodated within the lumen of the external sleeve and having a distal end which is engaged with the proximal end of the device and being in flow communication with the duct within said body; and an elongated actuation member accommodated within the inner sleeve for actuation, through axial displacement, of the device from the un-deployed to the deployed state.

In accordance with an embodiment of the invention said actuation member is tubular and has one or more engaging members at its distal end that engage with the said device in a manner such that axial displacement of the actuation member actuates the device to switch from an un-deployed to a deployed state; said actuation member accommodates a central rod that acts to secure the engagement of the one or more engaging members with said device, pulling of said central rod allowing disengagement of the one or more engaging members.

In accordance with one specific embodiment of the invention, a system for deployment of an anastomosis device of the kind having an internal and external body part, of a kind described above. The system according to the embodiment has the following functional states: an un-deployed state for inserting the device to its place of deployment within the body, in which said device is accommodated within the distal end of the external sleeve, the distal end of said inner sleeve engages the proximal end of the device, the engaging members at the distal end of the actuation member engage the internal body part of the device, and the central rod is fully inserted within the lumen of the actuation member whereby it secures the engagement of said engaging members to said internal body part; one or two first operational states in which the external sleeve is retracted exposing at least one of the tissue engaging units; corresponding one or two second operational states in which either the actuation member or the internal sleeve (or both) is axially displaced so as to cause opening of the exposed tissue engaging unit; a third operational state in which the internal body part is fully axially displaced in the proximal direction whereupon a continuous fluid path is formed between the internal duct of the device and the lumen of the inner sleeve; a fourth operational step in which the central rod is removed, whereupon the actuation member can be disengaged from the inner sleeve; and a resulting deployed state in which the actuation member is removed, the device is in its deployed state, the inner sleeve remaining engaged with the device serving for catheterization.

Typically, the first and the second operational states are repeated. In the first repeat, the external sleeve is retracted to expose a first tissue-engaging unit and then the actuation member or the internal sleeve (or both) is axially displaced to actuate the opening of the first unit. Then in the second pair the external sleeve is retracted further to expose a second tissue-engaging unit and the actuation member or the internal sleeve (or both) is axially displaced to actuate the opening of the second unit.

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIG. 1 depicts an anastomosis system for use in joining the bladder to the urethra comprising a deployment instrument and a tissue-fastening device carried thereon in accordance with an embodiment of the present invention;

FIG. 2A depicts the anastomosis system of FIG. 1 in an open state of the two tissue-engaging units;

FIG. 2B depicts the anastomosis system of FIG. 1 in an open state of the two tissue-engaging units after removal of the deployment mechanism;

FIG. 3 is a simplified schematic flowchart of a process for performing an anastomosis, in accordance with some embodiments of the present invention;

FIG. 4 depicts an isometric view of an anastomosis system of FIG. 1 deployed in situ showing a tissue-fastening device attached to a catheterization tube which originally constituted part of the deployment apparatus;

FIG. 5 is an isometric view of a tissue fastening device in accordance with an embodiment of the invention in its close state, detached from the deployment instrument;

FIG. 6 is an isometric view of the inner body part of the device of FIG. 5;

FIG. 7 is a longitudinal cross-section of the device of FIG. 5;

FIG. 8 is a longitudinal cross-section of the device of FIG. 5 in the opened state of the tissue-engaging units;

FIG. 9 is an isometric, partially exploded view of the device of FIG. 5 and the distal portion of the deployment instrument;

FIGS. 10A-10E are isometric views of the tissue-fastening device and the distal portion of the deployment instrument according to another embodiment in successive opening and deployment steps of the device;

FIGS. 11A-11E are longitudinal cross-sections of the device in a succession of deployment/opening steps corresponding to those of respective FIGS. 10A-10E;

FIGS. 11F-11G are longitudinal cross-sections showing two additional operational steps, in which FIG. 11F shows the disengagement of the puller sleeve while FIG. 11G shows the disengagement of the external and inner sleeves from the device.

FIG. 12 is an isometric view of the tissue-fastening device, according to an embodiment of the invention, fitted with a thread for use in removal of the device from its deployed position;

FIG. 13A-13C is an isometric view of a device according to an embodiment of the invention in three successive removal steps;

FIG. 14-16 are isometric views of tissue-fastening devices according to other embodiments of the invention;

FIG. 17A-17B depicts respective closed and open states of a device according to another embodiment of the invention;

FIG. 18 depicts an anastomosis device attached to a guide wire for guiding instrument thereto; and

FIG. 19 shows a deployed anastomosis device in accordance with another embodiment of the invention.

### DETAILED DESCRIPTION OF EXEMPLARY EMBODIMENTS

In the following description some embodiments will be illustrated with reference to the annexed drawings, which concern a tissue fastening device and anastomosis system for use in linking the bladder and the urethra, for example, following a surgical prostatectomy procedure. As will be appreciated, the illustrated embodiments are intended to provide examples for the much broader aspect of the invention as described and defined herein, and these should thus not be construed as limiting. For example, applications of the invention may be in uses such as in joining blood vessels, joining segments of the gastro-intestinal tract. Thus, in addition to urological applications, the invention may have applications in gynecology, gastro-enterology, cardiovascular medicine, and others.

In the following description, like reference numerals in different figures indicate components or part with a similar, at times identical, function.

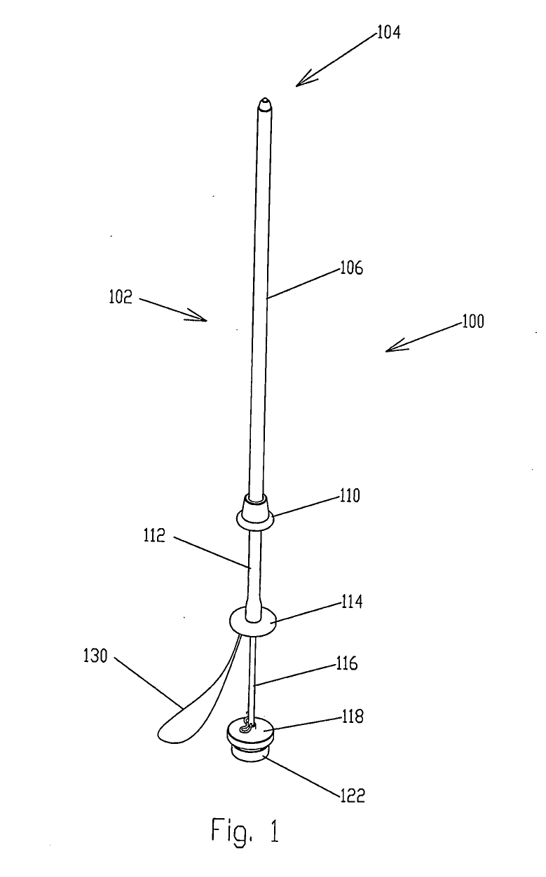

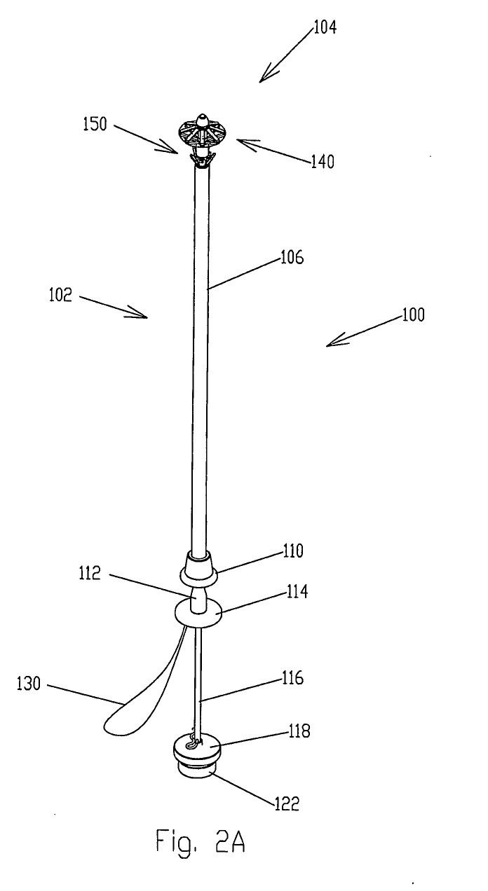

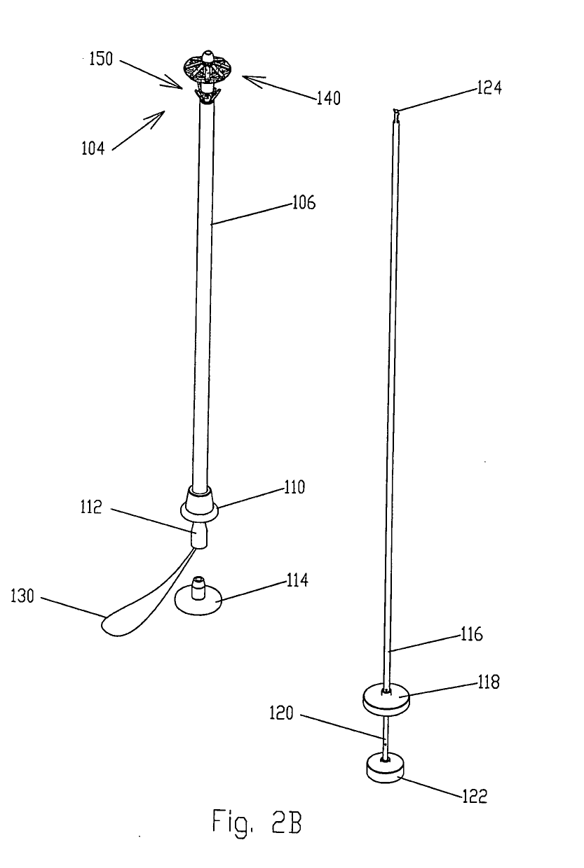

Reference is first being made to FIGS. 1, 2A and 2B showing an anastomosis system 100 including a deployment instrument generally designated 102 and a tissue fastening device 104 carried on the distal end of deployment instrument 102. Instrument 102 comprises an external sleeve 106 having a grip 110 at its proximal end, an internal sleeve 112 accommodated within sleeve 106 and having a grip 114 at its proximal end, a puller sleeve 116 fitted with a proximal grip 118 and a central rod 120 (that can be seen more clearly in FIG. 2B), ending with a proximal grip 122. Puller sleeve 116 has two engagement arms 124 at its distal end. Also shown is a device release thread 130 which extends from device 104 through sleeve 112 and out through the proximal end thereof. This string assists in removal of the device as will be explained further below.

Initially, as shown in FIG. 1, the device 104 carried at the end of deployment instrument 102 is in a closed or un-deployed state and almost entirely enveloped by the distal end of external sleeve 106. In a manner to be explained further below, device 104 can be actuated, through a series of manipulations of deployment instrument 102, whereupon tissue fastening units 140 and 150 open to assume the state as shown in FIGS. 2A and 2B.

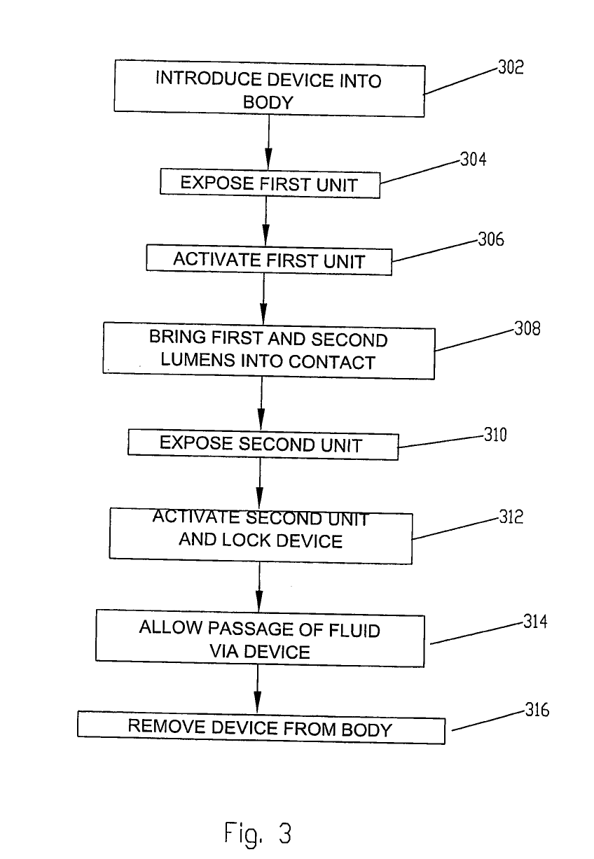

The series of operative steps in deployment of the tissue-fastening device 104 is presented schematically in FIG. 3. In the first step 302, the anastomosis device carried at the distal end of deployment device 102, is inserted through the urethra until device 104 lies partially within the bladder. At the next stage 304, through pulling of grip 110, while using grip 114 for a stabilizing bias, external sleeve 106 is retracted exposing first unit 140. At a next stage, 306, through pulling on grip 118, against the provision of a stabilizing bias on grip 114, puller sleeve 116 is retracted and in a manner to be explained below, causes the opening of unit 140 into a state as shown in FIG. 2B. Unit 140 is made to open within the bladder and consequently the walls of the bladder are pulled towards the urethra. If needed, at 308 the walls of the bladder may be pulled closer to the urethra by some further retraction of unit 140.

At a next stage 310, grip 110 is further pulled, thus causing additional retraction of external sleeve 106 thus exposing unit 150. By a further pulling on grip 118, sleeve 116 is retracted and through a mechanism, also to be explained below, unit 150 opens to its open position as shown in FIGS. 2A and 2B. Once unit 150 opens, the device is locked in the deployed state at 312, through a mechanism such as one of those to be described below, and the device remains locked in this position whereby units 140 and 150 remain open. The locking of the device in the deployed state should be such so as not to be destabilized and spontaneously close by normal biasing forces that may be exerted on the device once deployed in situ.

Device 104 has an internal duct (not shown in FIGS. 1-2B) which in the deployed and locked state 314 is in fluid communication with internal sleeve 112. In this deployed state, at 316 all components of the deployment device are removed other than sleeve 112 and external sleeve 106. The sleeve 112, which remains linked to device 104, serves for catheterization of fluids from the distal end of device 104 which is then drained out through the proximal end of sleeve 112 which can, be connected to a drainage bag or the like. The sleeve 112 remains linked to the internal body of the fastening device through the interdigitation between them and is constrained in this engagement due to the overlap of the external sleeve.

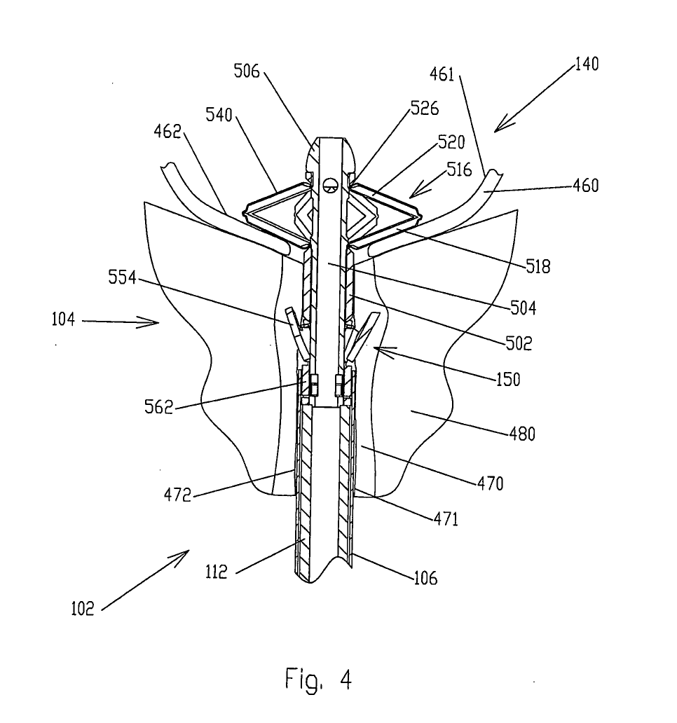

Reference is now being made to FIG. 4 showing an anastomosis device 104, according to an embodiment of the invention, for joining a bladder 460 with a urethral stump 470 and deployed for the purpose. The tissue-engaging device includes a tissue-engaging-unit 140 that consists of a plurality of ribs 516, that are arranged in a general axial symmetry, and which are associated with a membrane 540. Once in situ in a deployed and open state as shown, membrane 540 forms a fluid-tight contact with internal walls 461,462 of the bladder 460. As can further be seen in FIG. 4 once opened within the bladder and brought into close association with the internal walls 461,462 of the bladder 460, a fluid-tight contact between unit 140 and the internal walls of the bladder is thus formed. Device 102 has another tissue-engaging unit 140 with a plurality of pointed prongs 552 that at least partially penetrate inner walls 471,472 of urethra 470. The particulars of the device of this embodiment will be explained in further details below.

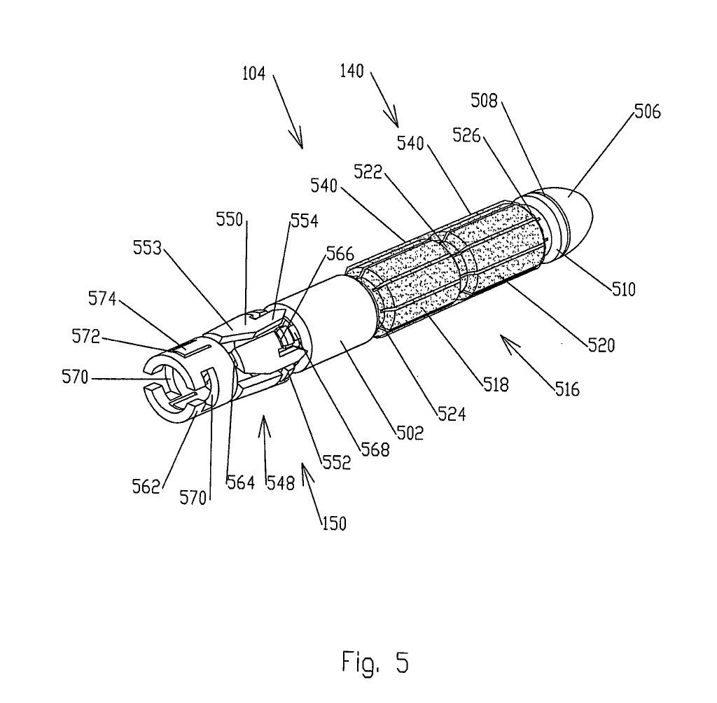

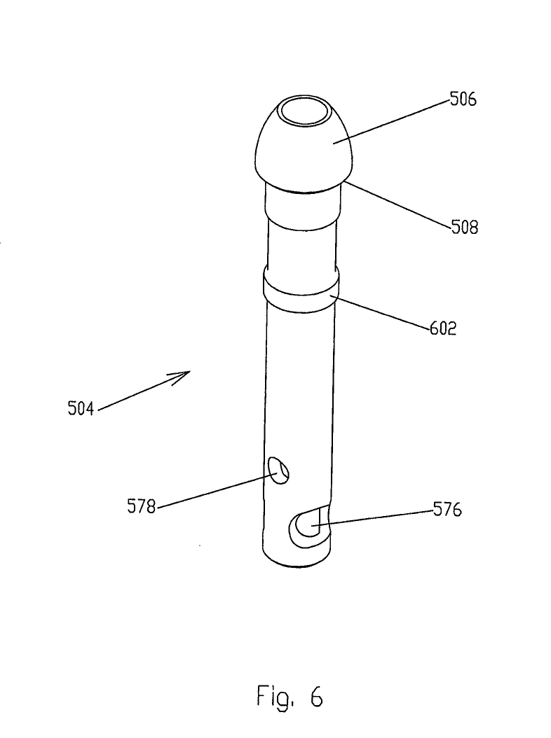

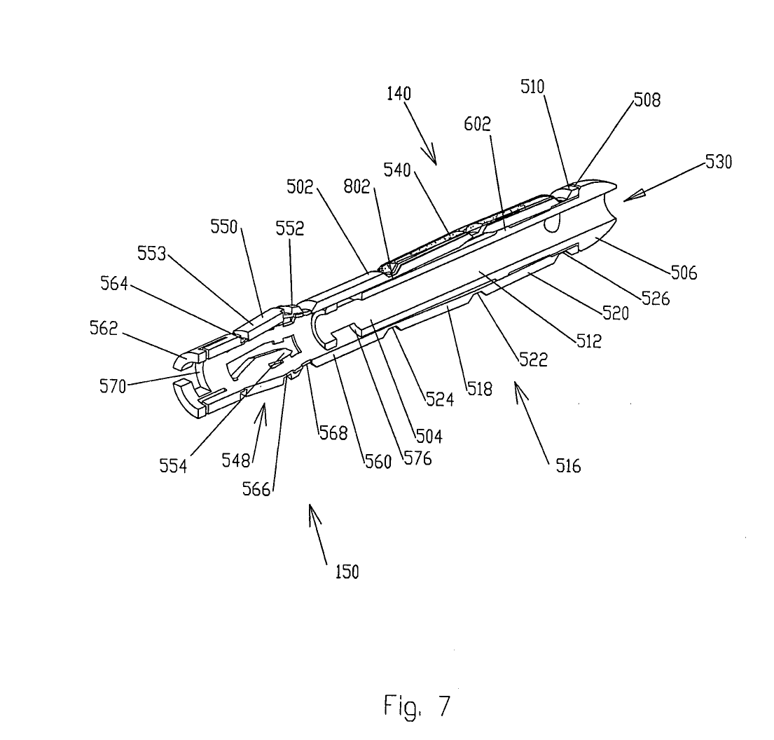

Reference is now being made to FIGS. 5-7 in which FIG. 5 is a perspective view of a tissue-fastening device according to an embodiment of the invention. FIG. 6 shows an internal sleeve of the device and FIG. 7 in a longitudinal cross-section. Device 104 has an overall cylindrically shaped body that includes an external tubular body part 502 and an internal tubular body part 504 accommodated within the lumen of body 502. Integrally formed within body part 502 are tissue-engaging units 140 and 150. Internal tubular body part 504 ends with a distal tapering head portion 506 that has shoulders 508 that engage the distal end portion 510 of external tubular body part 502. Defined within tubular body part 504 is a duct 512 defining a flow passageway through device 104.

Tissue-engaging unit 140 comprises a plurality of axially oriented ribs 516. Each of ribs 516 has a proximal segment 518 and a distal segment 520 pivotally linked to one another through a flexible connecting portion 522. Proximal segment 518 is pivotally linked to the proximal part of body 502 through a proximal flexible connecting portion 524, while distal segment 520 is pivotally linked through a distal flexible connecting portion 526 to distal end portion 510 of the external body part. Connecting portions 522, 524 and 526 form integral hinges and are formed with a relatively thinner wall which permits preferential bending at these locations. Overlaying the ribs is a membrane 540.

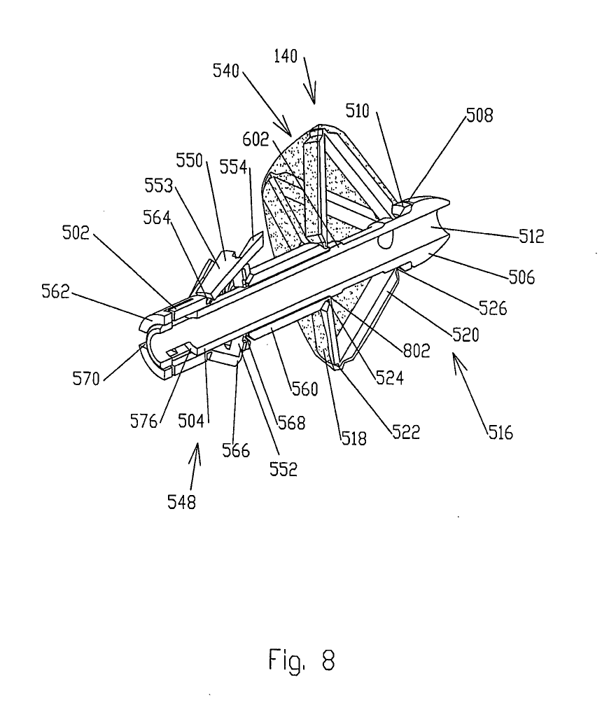

As will be appreciated, and as will also be shown below, upon displacement of internal body part 504 in the direction of arrow 530 (FIG. 7) relative to external body part 502, shoulder 508 will displace the distal body portion 510 giving rise to deployment of unit 140, by radial extension of ribs 516 to the state as shown in FIGS. 4, 8 and others. This deployment results from the opposite compression forces exercised on the external body of the fastener 502 within the deploying instrument; by pulling on the pulling sleeve 116 (not shown in these figures) that engage the internal body part 504, that latter engaging the distal rim of the external body part 510 by the shoulder 508, the net result being a force on the external body part 502 directed proximally which is against the stationary proximal end thereof. (The proximal end remains stationary in view of its engagement with internal sleeve 112 of the deploying instrument). Internal body part 504 has an annular abutment 602 and the rearward displacement is limited thereby and progresses until annular abutment 602 comes to bear against the base 802 of segment 518.

Unit 150 consists of a plurality of members 548, each with a proximal segment 550 and a distal segment 552. Segment 550 has a rear section 553 and a pointed tissue-penetrating prong 554. In FIG. 5, unit 150 is seen in an un-deployed state with the prongs being essentially level with the external surface of external body part 502 blending into the overall cylindrical structure. In the deployed state that can be seen in FIG. 8, pointed prongs 552 point outwardly and distally. In this position, they can penetrate the internal portion of the walls of the urethra, as can be seen in FIG. 4, and thus hold the walls in a fixed position. This improves approximation between the cut distal end of the urethra and the bladder neck.

Segment 550 is linked to the rear body portion 562 of external body 502 through a flexible proximal connecting portion 564 and to distal segment 552 through flexible intermediate connecting portion 566. Distal segment 552 is in turn linked to portion 560 of external body part 502 through a flexible connecting portion 568. Thus, in a similar mariner to the opening of the ribs of tissue-engagement unit 140, upon application of compression forces, as will be described below, tissue-engagement unit 150 opens to the state as seen in FIG. 8 in a manner to be described further below.

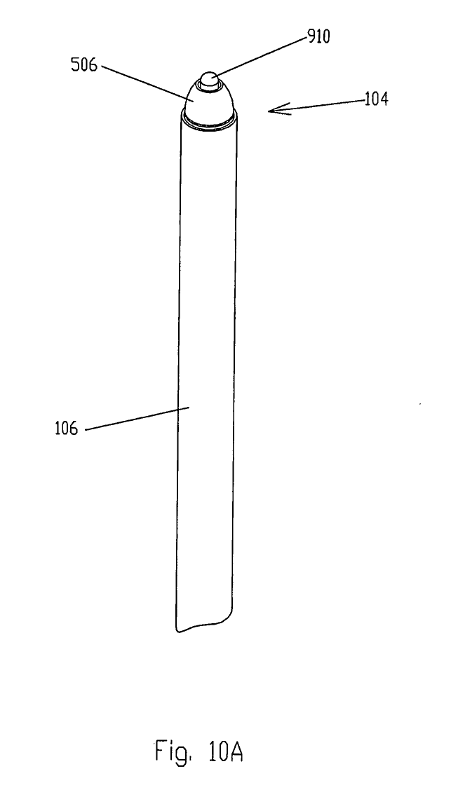

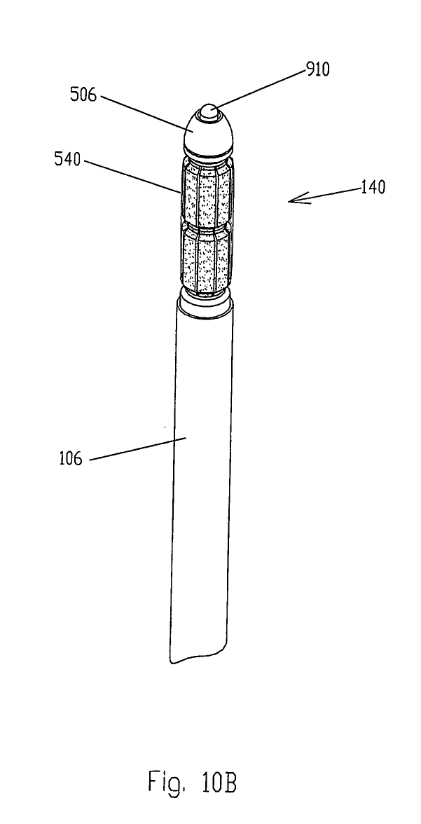

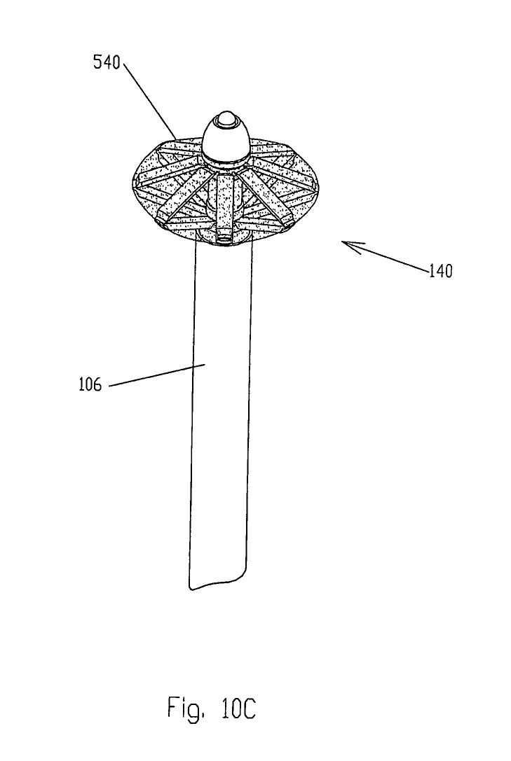

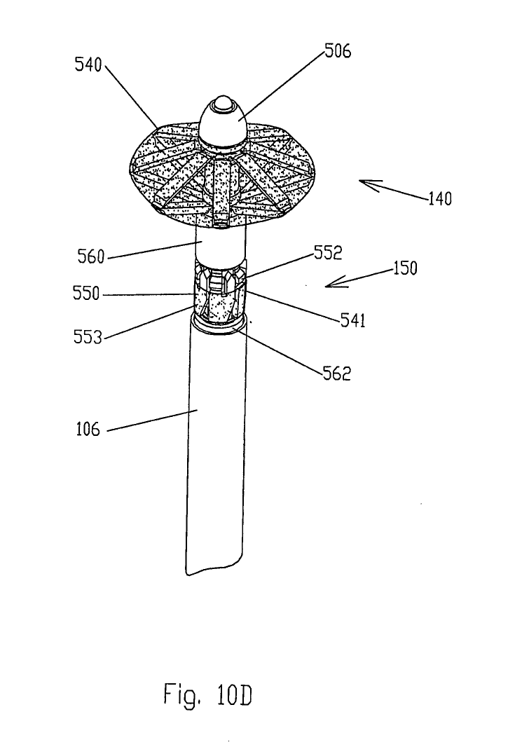

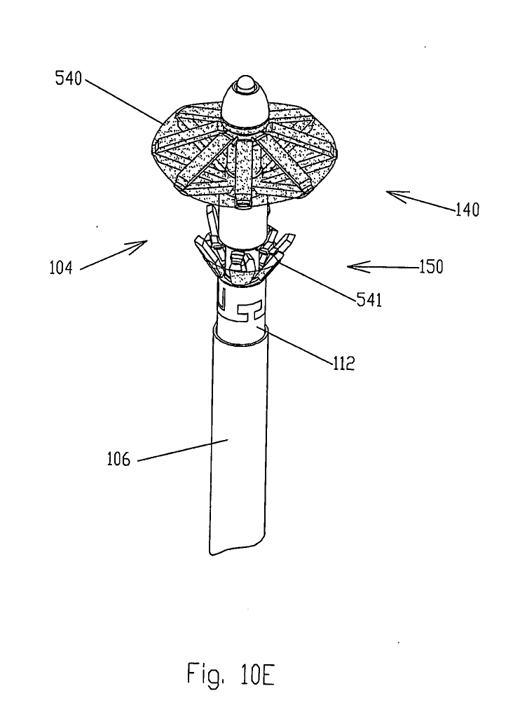

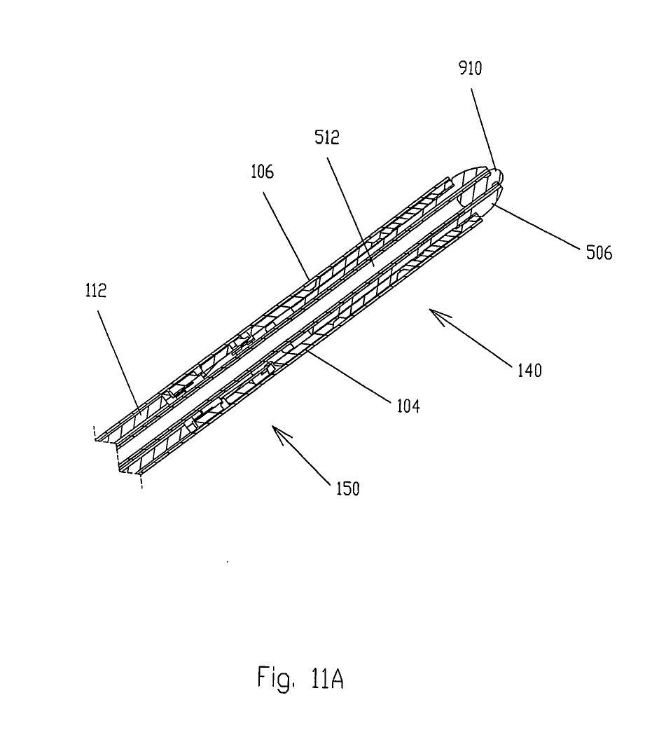

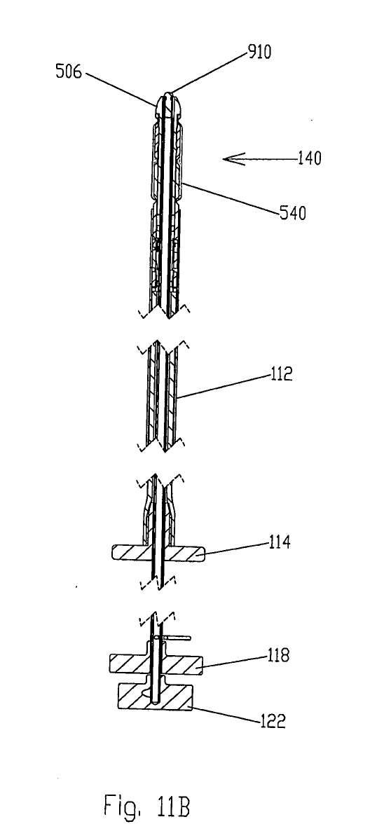

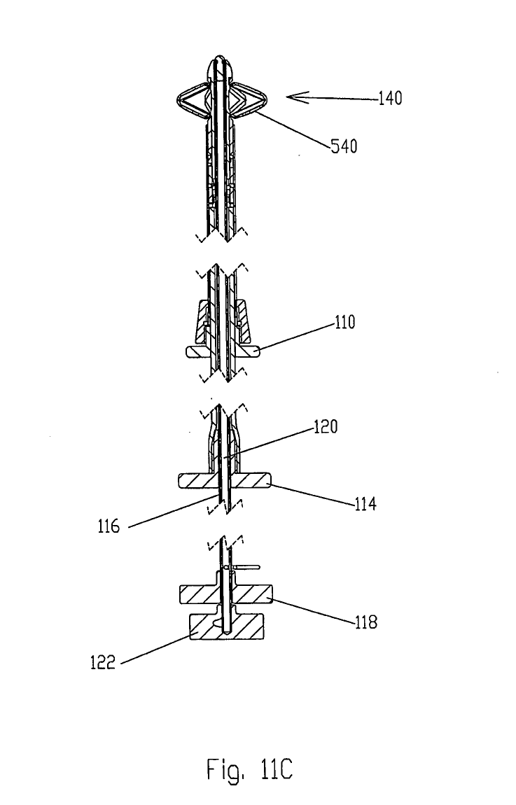

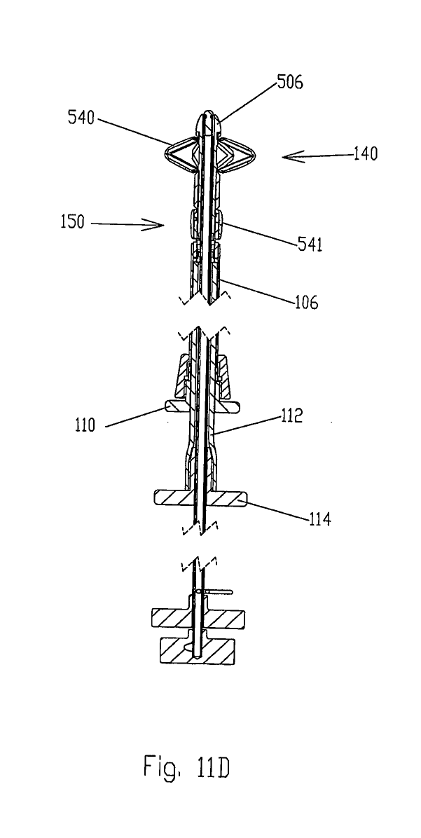

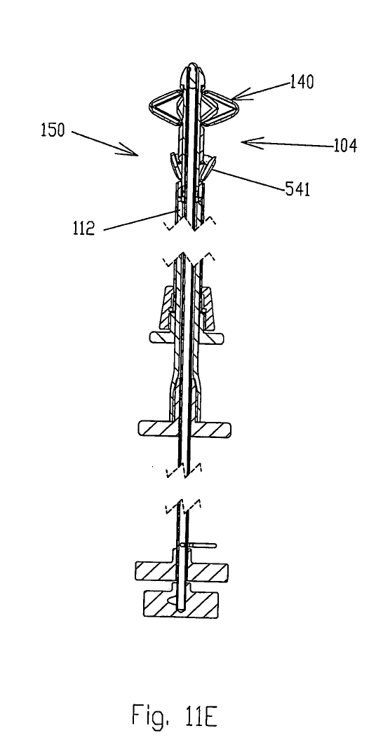

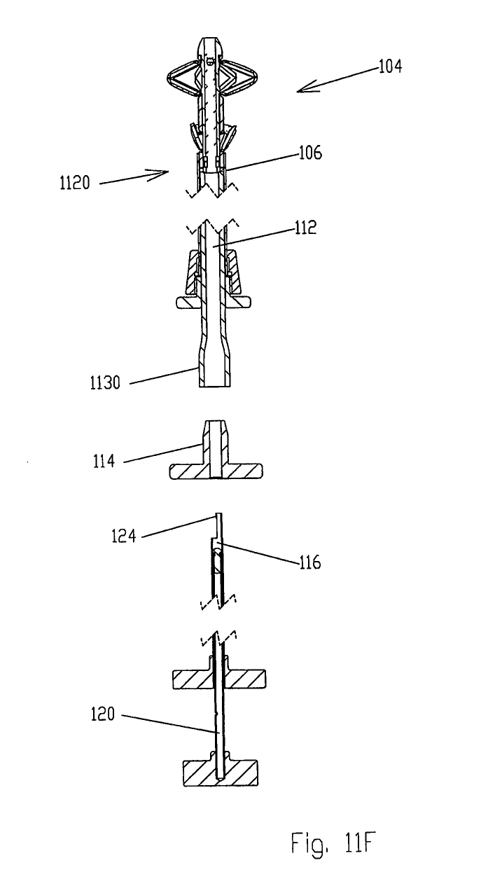

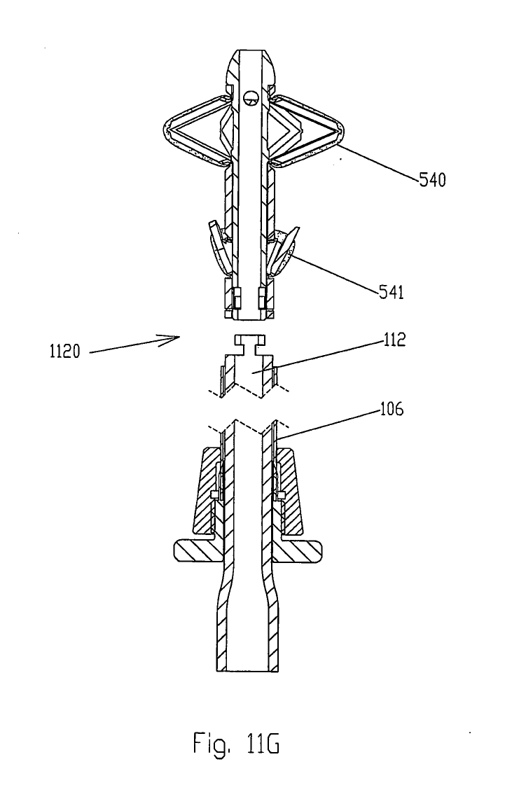

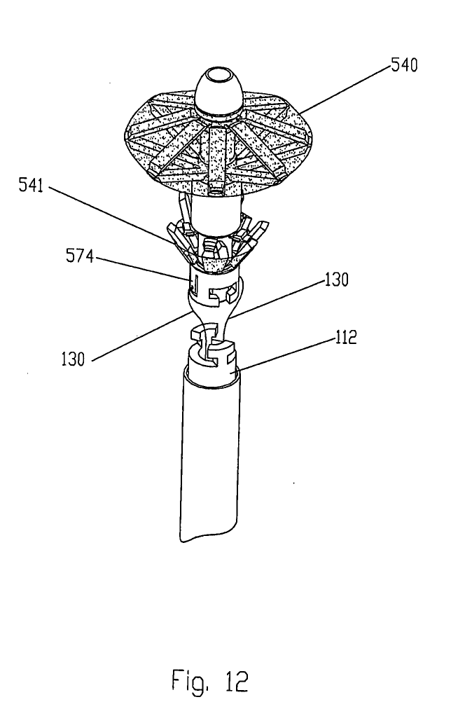

Optionally, in some embodiments of the invention, also tissue-engaging unit 150 is at least partially covered by a membrane (541, see FIGS. 10D-E, 11D-E, FIG. 11G and FIG. 12 herein below).

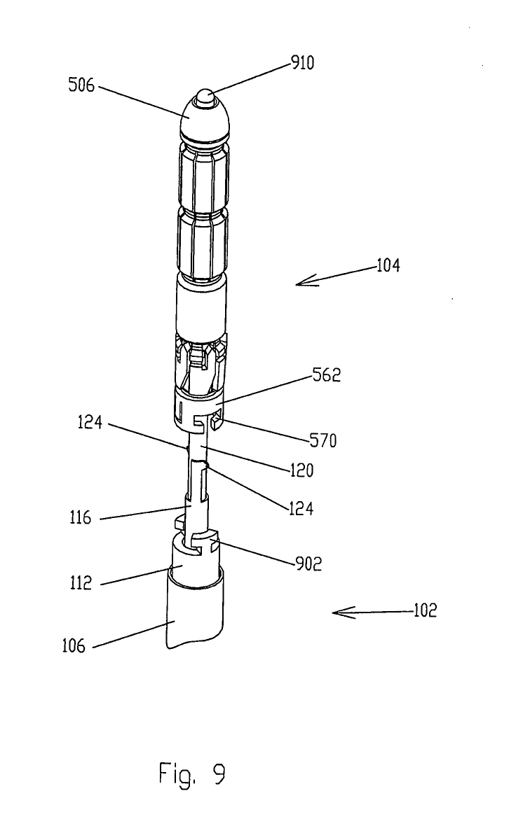

The proximal end 562 of body part 502 has a generally T-shaped groove 570 which serves for engagement of device 104 with the inner sleeve 112 (see FIG. 9 and the description also herein below).

Also provided in portion 562 of external tubular body part 502 are two engagement flaps 574. Flaps 574 are biased to slightly project internally and can thus engage with the openings 576 (seen in FIG. 6) of internal body part 504 to lock the device 104 in a fully deployed state as seen in FIG. 8. Alternatively, it is also possible, by other embodiments for flaps in the internal body part 504 to engage openings in the external tubular body part 502 to lock the fastener device in its fully deployed state. Another embodiment for engagement of the two body parts will be presented below.

Internal tubular body part 504 has also two lateral openings 578 (seen in FIG. 6) which are intended for engagement with distal engagement arms 124 of puller sleeve 116.

FIG. 9 shows a tissue fastening device 104 according to the embodiment depicted in FIG. 5. Also depicted in FIG. 9 are distal components of the deployment instrument. The view of FIG. 9 is partially exploded to permit clear view of the components of the deployment instrument.

As can be seen, inner sleeve 112 has a T-shaped engagement projection 902 which has a shape matching opening 570 and can thus be accommodated therein. This ensures a tight and continuous engagement of device 104 and inner tube 112. The engagement between inner sleeve 112 of deploying instrument and the external body part of the fastening device permits, among others, to exert a counterforce during deploying the device. Also, through such tight engagement, the inner sleeve 112 may serve as a catheter tube for fluid drainage after removing the pulling sleeve 112 and the internal rod 120, as already explained above, and as will also be explained further below.

Accommodated within the lumen of inner sleeve 112 is a puller sleeve 116 having terminal engagement arms 124 which engage with openings 578 of inner part 504 of device 104. Accommodated within puller sleeve 116 is inner rod 120 having a distal end 910 protruding slightly out of the distal opening of tapered head portion 506 of internal body part 504. As long as inner rod 120 passes between engagement arms 124, arms 124 are constrained to be in a laterally protruding position, firmly engaged within opening 578 of internal part 504 of device 104. As will also be explained below, the relative displacement of internal body part 504 and external body part 502 of device 104 is achieved by retraction of puller sleeve 116, which is facilitated by the tight engagement as aforesaid. Once rod 120 is retracted, engagement parts 124 can disengage from openings 578 of internal body part 504.

External sleeve 106 initially envelopes both inner tube 112 and device 104. Through the gradual retraction of sleeve 106 and by sequential actuation, as will be explained below in FIGS. 10A-11E, device 104 is opened and deployed.

FIGS. 10A-10E and 11A-11E describe a succession of steps for opening of the tissue engagement units while deploying device 104. Initially, sleeve 106 envelopes most of device 104 other than head portion 506. As can be seen, slightly protruding out of the opening of head portion 506 is the rounded end 910 of central rod 120. In the state as shown in FIGS. 10A and 11A, the distal part of the deployment system with the tissue fastening device is brought into position, e.g. in the case of intended anastomosis of the bladder and the urethra, is introduced through the urethra and advanced through the severed urethral stump and is inserted into the small pelvis, under vision. Then the instrument with said device is introduced into the urinary bladder through the severed bladder neck. At this stage, the sleeve 106 is retracted to the position as shown in FIGS. 10B and 11B and then the puller sleeve 116 is retracted by pulling at grip 118, jointly with central rod 120 and its grip 122, while counterforce is exercised on the internal sleeve 112 through grip 114; and through a mechanism as explained above causes opening of unit 140 to its open state as seen in FIGS. 10C and 11C.

At the next stage, sleeve 106 is retracted to the position as can be seen in FIGS. 10D and 11D in which unit 150 is exposed, and the entire deployment system is pulled so as to bring the severed stump of the bladder into closer proximity with the severed urethral stump. Upon further retraction of puller sleeve 116, while applying a counterforce on the internal sleeve 112 through grip 114 and through a mechanism as explained above unit 150 is opened to its open state as seen in FIGS. 10E and 11E. As can be seen, engagement unit 152 in this embodiment has an associated membrane 541 which is fitted over rear sections 553 of segments 550, and can thus also form, in its open state, a fluid-tight contact with surrounding walls.

As a final step shown in FIG. 11F, internal rod 120 is retracted relieving the constraint imposed on engagement arms 124 at the distal end of pulling sleeve 112 that engaged the internal body part 504. Then the puller sleeve 116 can be disengaged, retracted, and removed. The external sleeve 106 and inner sleeve 112 are left in place with the external sleeve covering the engagement zone 1120 between the inner sleeve 112 and the proximal portion 562 of the external body part of 502. This overlap between the external sleeve 106 and the engagement zone 1120 of the internal sleeve and the fastener device prevents disengagement of the internal sleeve from the anastomosis device. Grip 114 can then be removed leaving open the proximal end 1130 of the tube 112 that can now be connected to a urine removal system such as a urine bag

The inner sleeve 112 may be disengaged from the fastening device as represented in FIG. 11G by retracting the external sleeve 106 thus exposing the point of engagement 1120 of the internal sleeve with the external body part 502. The engagement at the point of engagement 1120 is secured through the stabilizing embrace of external sleeve 106 as it avoids transient deformation of the point of engagement 1120 which is needed for disengagement. Then by pulling on the internal sleeve 112, the internal sleeve can be easily disengaged from the deployed and locked fastener device and the internal sleeve 112 and external sleeve can be removed leaving the deployed fastening device as a stent for the anastomosis.

The role of the non-stretchable or minimally stretchable covering membrane is not only to provide sealing and prevent fluid leakage. The additional role is to increase the tangential stability of the deployed ribs. The membrane has to be attached to each rib and when fully deploying the ribs, the membrane is extended to its maximum and induces maximal radial and tangential stability of the tissue-engaging units covered by these membranes.

FIG. 12 shows another embodiment in which use is made of a release thread 130 that is linked at its distal end to engagement flaps 574 and extends through the lumen of inner sleeve 112 out through its proximal end as shown in FIGS. 1-2B. Threads 130 are affixed to flaps 574 at their exterior. Upon pulling thread 130, flaps 574 are slightly radially displaced thereby disengaging openings 576 in internal body part 504.

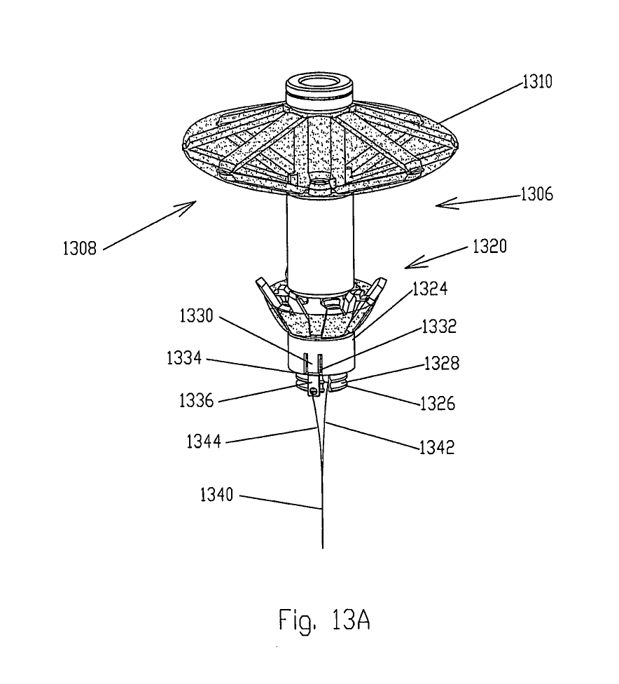

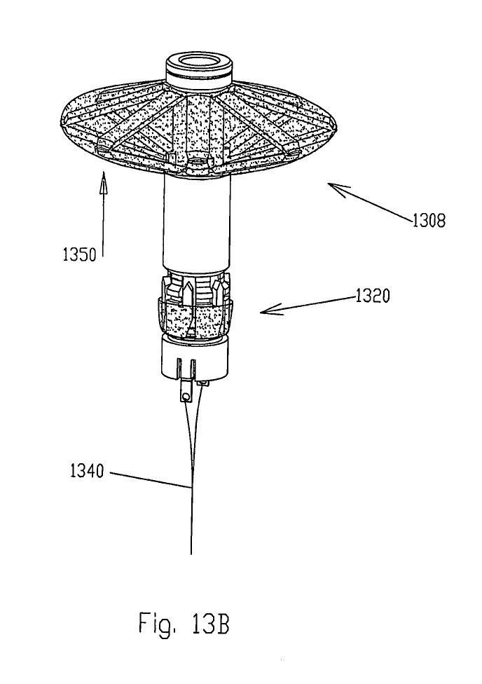

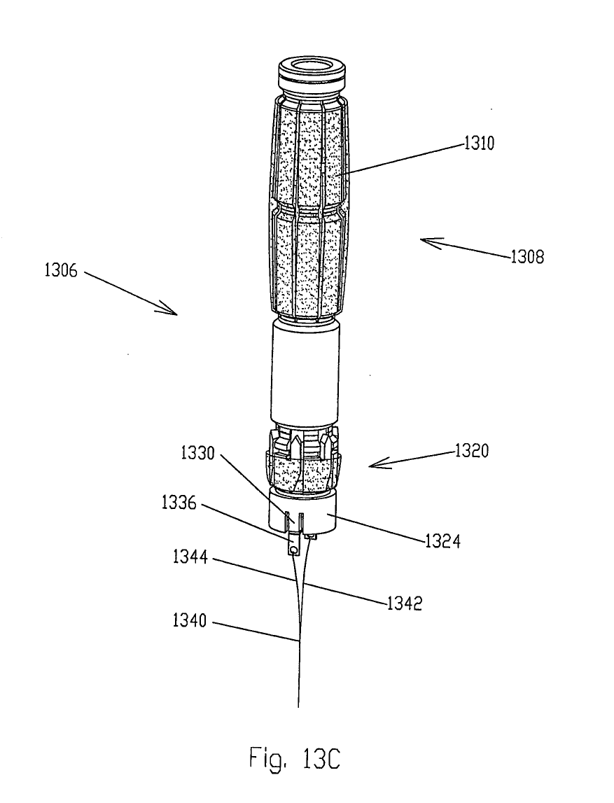

A succession of steps of un-deployment of an anastomosis device 1306 according to another embodiment of the invention is shown in FIGS. 13A-13C. This device also displays an alternative engagement mechanism between the two body parts than that described above. Device 1306 according to this embodiment has a tissue-engaging unit 1308, of substantially similar design to tissue-engaging unit 140 in the embodiment described above. Engagement unit 1308 has an associated membrane 1310. Axially disposed in the proximal direction is a second tissue-engaging unit 1320 which is substantially similar to the tissue-engaging unit 150 in the embodiment described above.

The proximal portion 1326 of the internal body part is seen in FIG. 13A extending out from the proximal end 1324 of the device. Rear end 1326 has annular grooves 1328. This rear end serves for engagement in a ratchet-type mechanism. Flap 1330 is formed in the rear end of the external body part 1324 and has an internal abutment 1332 which engages one of the grooves and locks the device in a deployed state. The groove external surface of the proximal end 1326 of the device's inner body part also serves for engagement with the distal end of inner sleeve 112.

Flap 1330 is linked through a flexible connecting portion 1334 (which has a relatively thin wall similarly to the above described flexible connecting portions) to an extension flap 1336. Extension flap is connected to thread 1340 through bifurcating terminal segments 1342 and 1344, one connecting to extension flap 1336 and the other to its counterpart at the other end.

Pulling of thread 1340 causes a slight axial displacement of flap 1330 whereupon abutment 1332 disengages the corresponding groove 1328 releasing the internal body part and permitting closure of tissue-engagement unit 1320 to the closed state as seen in FIG. 13B. The additional pulling exerts a distally directed force as represented by arrow 1350 in FIG. 13B, on tissue-engaging unit 1308 causing its closure to the fully closed state, seen in FIG. 13C, and at this un-deployed state the device can be retracted from its position.

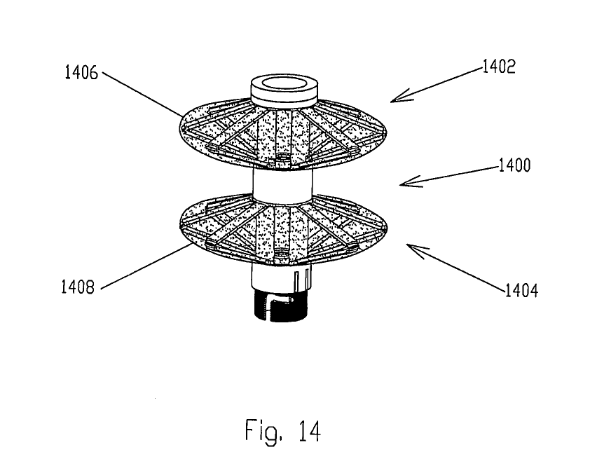

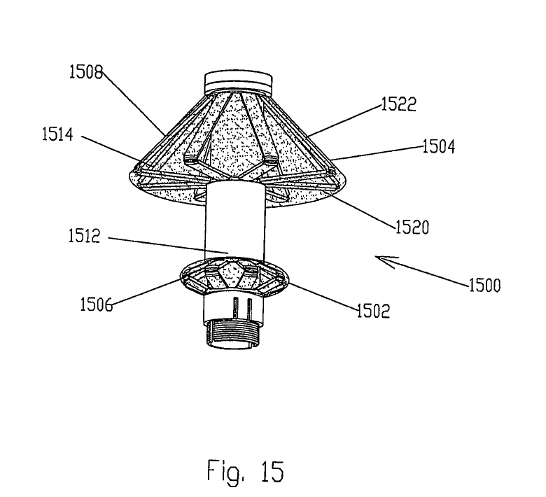

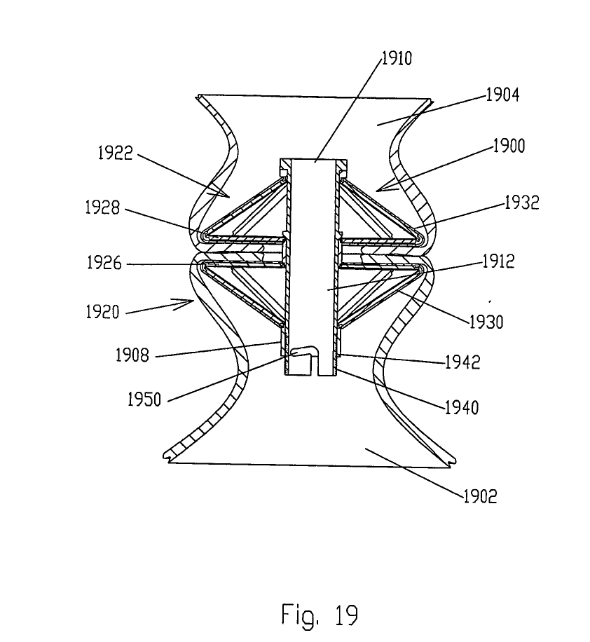

FIGS. 14-16 show different embodiments of design of an anastomosis device in accordance with the invention. Device 1400 seen in FIG. 14 has two essentially identical tissue-engaging units consisting of a distal unit 1402 and a proximal unit 1404, each of which has ribs 1406 and an associated membrane 1408, similar to that of unit 140 of the embodiment described above. Device 1400 according to this embodiment is suitable for anastomosis of tubular organ of a similar cross section, for example, two cut stumps of blood vessels, two cut stumps of intestine, etc. In some cases, units 1402,1404 are only partially covered by a membrane 1408. Device 1400, as well as device 1500 as seen in FIG. 15, have a ratchet-type engagement mechanism for locking the device in the deployed state of a kind described in the embodiment of FIGS. 13A-13C and in the embodiment of FIG. 19, that will be described further below.

A device 1500 according to another embodiment of the invention is shown in FIG. 15. The device has a relatively small, proximal engagement unit 1502 and a distal larger engagement unit 1504. Both units consist of respective ribs 1506 and 1508 and are associated with respective membranes 1512 and 1514. Tissue engagement unit 1502 opens to an essentially symmetrical configuration while unit 1504 comprises ribs that have each a proximal segment 1520 and a distal segment 1522 which is longer than the former whereby segments 1520 assume an essentially right angle with respect to the axis of the device 1500.

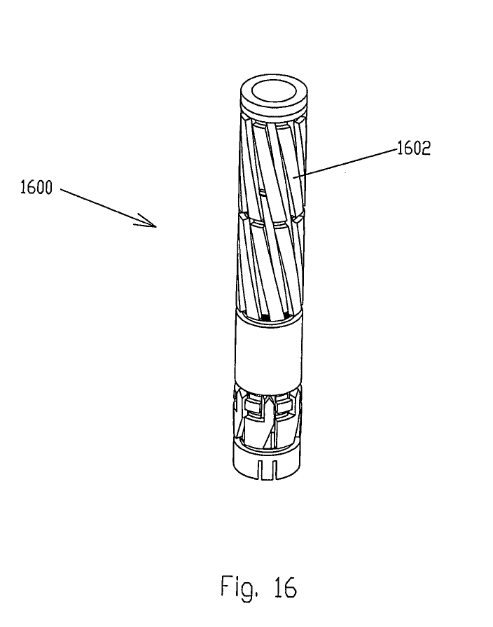

A tissue fastening device 1600 according to another embodiment of the invention is seen in FIG. 16. In this case, the ribs 1602, rather than having an axial orientation in their closed state, have a somewhat slanted orientation in the closed state.

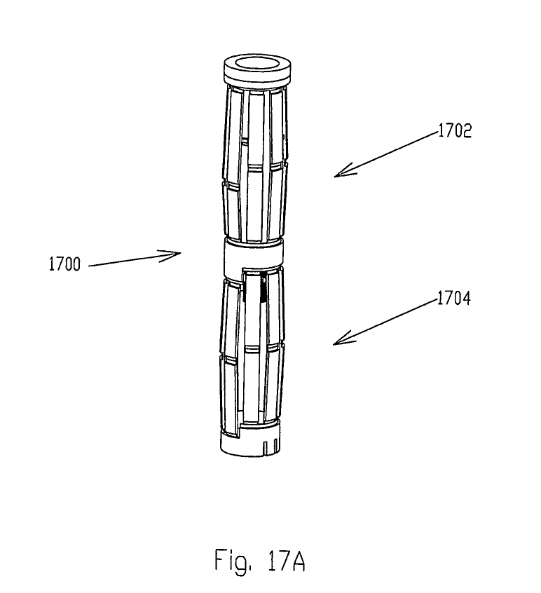

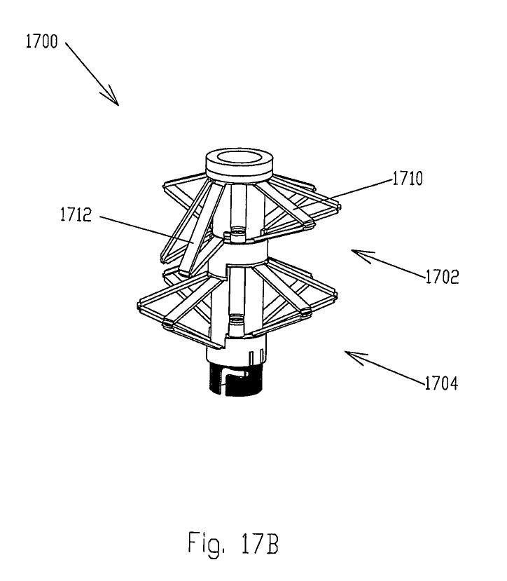

A tissue fastening device according to another embodiment of the invention is shown in a closed and open state in FIGS. 17A and 17B. Device 1700 has two tissue engagement units 1702 and 1704, each of which has a plurality of ribs with a similar general structure as in the case of device 104, however, with some asymmetric features. In the case of unit 1702, the different ribs differ from one another with the relative length of their proximal and distal segments whereby upon opening, some of the ribs 1710 assume a general symmetric open shape, others 1712 assume an asymmetric open shape.

In unit 1704, the plurality of ribs are all designed to have a symmetric opened shape, however, different ribs have a different axial position in the device whereby the overall shape of the unit in its open shape is non symmetrical. Thus, a fastening device of this nature may be used to fasten two organs having irregular shape.

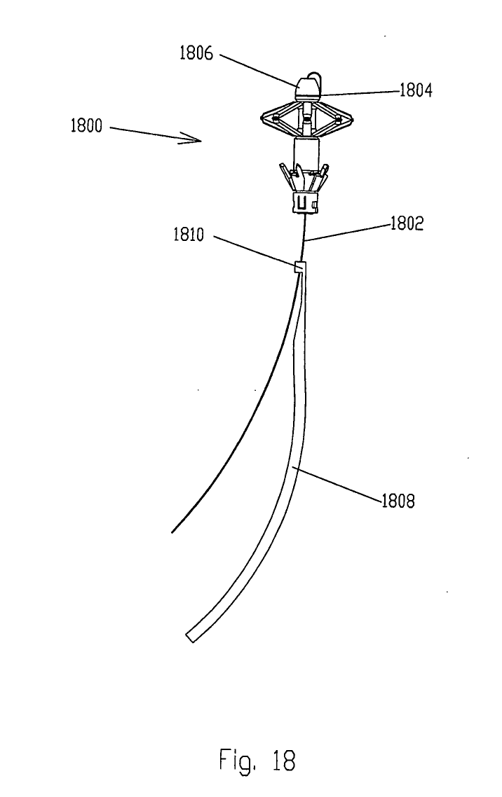

Reference is now being made to FIG. 18, showing an anastomosis device 1800 with a general structure as device 104 described above. Device 1800 is fitted with a guide wire 1802 which passes through the internal duct of the device and fastens at its distal end 1804 to the distal end 1806 of the inner body part. This guide wire permits guidance of components of the deployment or un-deployment instruments, such as device-engaging member 1808 which has a distal tubular portion 1810 which thereby guides the distal end of device 1808 into position.

Reference is made to another embodiment of an anastomosis device 1900 which is seen in FIG. 19, in a deployed state linking together two stumps consisting of a proximal stump 1902 and a distal stump 1904 of a tubular body part for example, two stumps of a cut intestine. Unit 1900 which is seen in longitudinal cross-section, consists of an external body part 1908 and an internal body part 1910 which can be axially displaced one with respect to the other in a manner analogous to that described above. Internal body part has a lumen 1912 extending therethrough.

Device 1900 has two tissue-engaging units—a proximal tissue-engaging unit 1920 and a distal, tissue-engaging unit 1922, which in the open state as seen in FIG. 19 have each respective distal segments 1926 and proximal segments 1928 which have a generally right angle orientation with respect to the axis of the device, similarly, as in the case of the tissue-engaging unit 1504 of device 1500 as seen in FIG. 15. Covering each of tissue-engaging units 1920 and 1922 are respective membranes 1930 and 1932. Thus, in the fully open and deployed state seen in FIG. 19, the two tissue-engaging units clamp wall portions between them in a fluid tight manner.

As can be seen, the proximal end 1940 of inner body part 1910 has a grooved surface with a saw-cross-sectional shape whereby it can engage with an annular internal terminal abutment 1942 in the proximal end of the external body part 1908 in a ratchet-type engagement. As can be appreciated, given this engagement mechanism, this device has several locking states rather than a single locking state as, for example, in the device 104 described above.

The inner body part 1910 has a generally L-shaped groove 1950 which serves for engagement with a deployment instrument.

According to some embodiments of the present invention, the deployment instrument is typically made out of a metal or alloy, such as stainless steel, or made of a plastic material or a combination thereof. The external sleeve may be made of a metal or of a plastic material, depending on the application requirements.

The deploying instrument may be straight, bended, rigid, elastic, or deflectable.

The internal sleeve, which, once deployed, serves as a catheter tube, may be made of plastic, polypropylene polyurethane, polyethylene, silicone, or any other flexible material.

The device may be made of a plastic material, nitinol, or of bio-erodible or biodegradable material and may, according to some embodiments, be left in situ until it is fully eroded and/or degraded. Additionally, only some of its components such as the locking mechanism may be made of a biodegradable material.

In some alternative embodiments, the bio-erodible/biodegradable device may be left within the anastomosis until its structural strength weakens sufficiently so that at least a part of the device, such as a blocking mechanism, softens to an extent such that by pulling on the connected thread, the prongs will be pulled out easily from tissue and device will be pulled easily from the urethra or the device will be spontaneously passed through the urethra.

Additionally, the device may be designed of some biodegradable components such as the prongs and/or the blocking elements and other non-biodegradable elements.

Alternatively, the device may be designed of a biodegradable material reinforced with a backbone of soft non biodegradable material. In this case the hinges of the flanges may consist of that soft backbone. Biodegradation of the attachment device will result in softening of the device but its structural integrity will be maintained assuring its integral removal.

The membrane may be made of a waterproof or water-resistant material, such as a polymer, exemplified by polyurethane, polypropylene, silicone, latex, and others.

1. An anastomosis device for associating two lumen-comprising body structures, having an un-deployed and a deployed state and switchable from the former to the latter, the device comprising:
a body with an overall cylindrical shape defining a proximal-distal extending axis of and comprising an external essentially cylindrical body part with a lumen and an internal body part at least a portion thereof being deployed within the lumen of the external body part and axially displaceable with respect thereto in the switch between the un-deployed and the deployed states; said internal body part having an internal duct extending axially therethrough whereby in the deployed state there is a fluid communication between the distal and the proximal ends of the body; and
two tissue-engaging units axially displaced from one another along said axis; at least one of the two units can change its state from a closed state in the un-deployed state of the device to an opened, tissue-engaging state in the deployed state of the device in which said first unit extends in an overall radial direction and is adapted in this state for tight association with internal walls of one of the body structures, such that the combined tissue-engaging action of the two unit holds the two structures together.

2. An anastomosis device according to claim 1, wherein said internal body part is retracted in the proximal direction with respect to the external body part when the device is switched from the un-deployed to the deployed state.

3. An anastomosis device according to claim 1 or 2, switchable also from a deployed state to an un-deployed state.

4. An anastomosis device according to any one of claims 1-3, wherein the external body part has one or more first engaging members, and the internal body part has one or more second engaging members, the one or more first engaging members engage the one or more second engaging members in the deployed state of the device, to lock the device in this state.

5. An anastomosis device according to claim 4, wherein the one or more first engaging members can be disengaged from the one or more second engaging members for switching the device from the deployed to the un-deployed state.

6. A device according to any one of claim 4 or 5, comprising a mechanism for disengaging the internal from the external body part for un-deployment of the device.

7. The device of claim 6, wherein the disengaging mechanism comprises one or more threads such that through pulling on these threads the engagement is released.

8. The device of claim 1 wherein at least the locking means consists of a biodegradable or a bioerodable material and biodegradation or bioerosion of these components releases the engagement.

9. An anastomosis device according to any one of claims 1-8, wherein at least one of the two tissue-engaging units is an integral unit integrally formed with the external body part.

10. An anastomosis device according to claim 9, wherein said integral unit is formed such that in its closed state it blends into the overall cylindrical shape of the external body part and comprises engaging members that are each pivotally anchored to rigid portions of the external body part through a flexible connecting portion and extend in a general radial orientation in the open state.

11. An anastomosis device according to claim 10, wherein said integral unit comprises a plurality of ribs which in the closed state are folded and oriented in a general axial orientation and have external and internal faces, and comprises a membrane fitted over the internal or external faces.

12. An anastomosis device according to claim 10 or 11, wherein said integral unit comprises a membrane fitted on an internal or external faces of the ribs and at least partially covering said unit in its open state, whereby in said opened state the at least one of said units can form an essentially fluid-tight contact with the internal walls of said body structure.

13. An anastomosis device according to any one of claims 10-12, wherein the membrane is impregnated with an anti-microbial substance.

14. An anastomosis device according to any one of claims 11-13, wherein said membrane is non- or minimally stretchable and is attached to each one of the members and is stretched to maximum in the open state conferring axial and tangential stability and rigidity to the tissue engaging unit to which it is attached.

15. An anastomosis device according to any one of claims 1-11, wherein
said external body part has one or two sections defining an integrally formed tissue-engaging unit, each such unit comprising a plurality of engaging members each of which having a proximal and a distal segment linked to the external body part through respective proximal and distal flexible connecting portions and linked to one another through respective intermediate flexible connection portion; and
the internal body part having a portion that engages the distal end of the external body part such that axial displacement of the internal body part towards the proximal end with respect to the external body part exerts an axial force on said distal end and causes its axial displacement in the same direction, whereupon the said members radially opens into an open state in which the two segments are angled with respect to one another about the intermediate connecting portion and being each oppositely angled with respect to the external body part about the respective proximal and distal connecting portions.

16. An anastomosis device according to claim 15, wherein said tissue-engaging units blend into the overall cylindrical shape of the external body part in their closed state.

17. An anastomosis device according to claim 15 or 16, wherein
at least one of the engaging-units has a structure that comprises a plurality of elongated members extending in a general axial orientation, each of which comprises a proximal and a distal segment linked to one another through the intermediate flexible connecting portion at their respective distal and proximal ends, whereby in the open state the two segments define an overall triangular shape with an apex constituted by said intermediate connecting portion.

18. An anastomosis device according to claim 18, wherein said membrane is non- or minimally stretchable and is attached to each one of the ribs and is stretched to maximum in the open state conferring axial and tangential stability and rigidity to the tissue engaging unit to which it is attached.

19. An anastomosis device according to any one of claim 17-19, wherein the engaging units have each said structure.

20. An anastomosis device according to claim 20, wherein the two units are essentially identical.

21. An anastomosis device according to claim 20, wherein the two units have different size or shape.

22. An anastomosis device according to any one of claims 15-22, wherein
at least one of the engaging-units has a plurality of members with one segment linked at a point thereof between its proximal and distal ends to the other segment, said one segment having a free end with a pointed, tissue-penetrating tip, whereby in the open state, said free ends have an outwardly slanted orientation and can associate with internal walls of a body structure by penetrating inner layers thereof.

23. An anastomosis device according to claim 23, where said one segment is the proximal segment, whereby in the open state said free end points in general distal direction.

24. An anastomosis device according to any one of claims 1-24, wherein said tissue-engaging units have an essential axial symmetry.

25. A device according to any one of claims 1-22, wherein one of the tissue-engaging units has a fixed structure or is an inflatable balloon.

26. A device according to claim 11, wherein the distal segments and proximal segments of the engaging members are of essentially the same length.

27. A device according to claim 11, wherein the distal segments and proximal segments of the engaging members are of the different length.

28. A device according to claim 11, wherein the dimensions of the distal segments and proximal segments is different for different tissue-engaging members.

29. A device according to claim 11, wherein one or more engaging members of at least one of the tissue engaging units are positioned at different axial positions with respect to other engaging members of the same unit.

30. A device according to any one of claims 1-30, for use in anastomosis of a bladder to a urethra.

31. A device according to any one of claims 1-30, for use in anastomosis of sections of the gastro-intestinal tract or of a blood vessel.

32. An anastomosis device having an un-deployed and a deployed state and switchable from the former to the latter, the device comprising:
a body with an overall cylindrical shape defining a proximal-distal extending axis and two tissue-engaging units axially displaced from one another along said axis;
at least one of the two units can change its state from a closed state in the un-deployed state of the device to an opened, tissue-engaging state in the deployed state of the device in which said first unit extends in an overall radial direction and is adapted in this state for tight association with internal walls of one of the body structures, such that the combined tissue-engaging action of the two unit holds the two structures together;
said at least one unit having a membrane associated therewith such that in the tissue engaging state said membrane forms a fluid-tight contact with internal walls of the body structure.

33. An anastomosis device according to claim 32, wherein said body has an overall cylindrical shape defining a proximal-distal extending axis of and comprising an external essentially cylindrical body part with a lumen and an internal body part at least a portion thereof being deployed within the lumen of the external body part and axially displacable with respect thereto in the switch between the un-deployed and the deployed states; said internal body part having an internal duct extending axially therethrough whereby in the deployed state there is a fluid communication between the distal and the proximal ends of the body.

34. A device according to claim 33 or 34, wherein said at least one tissue-engaging unit is integrally formed with said body.

35. A device according to claim 34, wherein said tissue-engaging unit is integrally formed with an external body part.

36. An anastomosis device having an undeployed and a deployed state and switchable from the former to the latter, the device comprising:
an essentially cylindrical body defining a proximal-distal extending axis of and having an internal duct extending axially therethrough providing fluid communication between the distal and the proximal ends of the body;
an actuation mechanism for switching between the un-deployed and the deployed state of the device; and
two tissue-engaging units axially displaced from one another along said axis; at least one of the engaging units has a plurality of members each comprising a segment having a free end with a pointed, tissue-penetrating tip, in the un-deployed state of the device said members blend into the cylindrical shape of the body, and in the deployed state, said free ends have an outwardly slanted orientation and can associate with internal walls of a body structure by penetrating inner layers thereof.

37. A device according to claim 36, wherein said free ends point in distal direction, whereby in the deployed state said ends are outwardly and distally orientated.

38. A device according to claim 36 or 37, wherein at least one of the tissue-engaging units is integrally formed in the external body part.

39. A device according to any one of claims 36-38, wherein at least one of the tissue-engaging units has a membrane associated therewith such that in the tissue engaging state said membrane forms a fluid-tight contact with internal walls of the body structure.

40. An anastomosis system comprising a deployment instrument and a device according to any one of claims 1 to 39.

41. A system according to claim 40, wherein the deployment instrument comprises a tubular body that after deployment of the device remains attached thereto and serves for catheterization.

42. A system according to claim 40 or 41, wherein the deployment instrument comprises
an external tubular sleeve having a lumen which in the un-deployed state accommodates the device in its proximal end;
an inner sleeve accommodated within the lumen of the external sleeve and having a distal end which is engaged with the proximal end of the device and being in flow communication with the duct within said body; and
an elongated actuation member accommodated within the inner sleeve for actuation, through axial displacement, of the device from the un-deployed to the deployed state.

43. A system according to claim 42, wherein said actuation member is tubular and has one or more engaging members at its distal end that engage with the said device in a manner such that axial displacement of the actuation member actuates the device to switch from an un-deployed to a deployed state; said actuation member accommodates a central rod that acts to secure the engagement of the one or more engaging members with said device, pulling of said central rod allowing disengagement of the one or more engaging members.

44. A system according to claim 43, for deployment of a device according to any one of claims 1-31, having:
an undeployed state for inserting the device to its place of deployment within the body, in which said device is accommodated within the distal end of the external sleeve, the distal end of said inner sleeve engages the proximal end of the device, the engaging members at the end of the actuation member engage the inner body part of the device, and the central rod is fully inserted within the lumen of the actuation member whereby it secures the engagement of said engaging members to said inner body part;
one or two first operational states in which the external sleeve is retracted exposing at least one of the tissue engaging units;
corresponding one or two second operational states in which either one or both of the actuation member and the internal sleeve is axially displaced so as to cause opening of the exposed tissue engaging unit;
a third operational state in which the internal body part is fully axially displaced in the proximal direction whereupon a continuous fluid path is formed between the internal duct of the device and the lumen of the inner sleeve;
a fourth operational step in which the central rod is removed, whereupon the actuation member can be disengaged from the inner sleeve; and
a resulting deployed state in which the actuation member is removed, the device is in its deployed state, the inner sleeve remaining engaged with the device serving for catheterization.

45. A system according to claim 44, wherein the first and second operational steps are repeated, in the first repeat the external sleeve is retracted to expose a first tissue-engaging unit and the actuation member is axially displaced to actuate the opening of the first unit; and in the second repeat the external sleeve is retracted further to expose a second tissue-engaging unit and one or both of the actuation member or the internal sleeve is axially displaced to actuate the opening of the second unit.
